(19) United States
(12) Patent Application Publication
(10) Pub. No.: US 2020/0043583 A1
(43) Pub. Date: Feb. 6, 2020

(54) SYSTEM AND METHOD FOR WORKFLOW-SENSITIVE STRUCTURED FINDING OBJECT (SFO) RECOMMENDATION FOR CLINICAL CARE CONTINUUM

(71) Applicant: KONINKLIJKE PHILIPS N.V., EINDHOVEN (NL)

(72) Inventors: Gabriel Ryan MANKOVICH, Boston, MA (US); Amir Mohammad TAHMASEBI MARAGHOOSH, Arlington, MA (US)

(21) Appl. No.: 16/338,530

(22) PCT Filed: Oct. 13, 2017
(86) PCT No.: PCT/IB2017/056339

§ 371 Date: Apr. 1, 2019

Related U.S. Application Data
(60) Provisional application No. 62/408,876, filed on Oct. 17, 2016.

(51) Int. Cl. G16H 15/00; G16H 30/40; G16H 10/60; G06F 17/24; G06F 17/28

(52) U.S. Cl. CPC G16H 15/00; G16H 30/40; G06F 17/2881; G06F 17/243; G16H 10/60

(57) ABSTRACT

A report-entry workstation (10) includes a computer with a display (14, 16), one or more user input devices (20, 22, 24), and at least one processor (1) programmed to operate the computer to perform operations. The operations includes: providing a user interface (18) enabling a logged in user of the workstation to operate the one or more user input devices to retrieve and view, on the display, images (26) of a radiological imaging examination stored in a radiology database (12) and to operate the one or more user input devices to enter a medical report (30); receiving data related to the logged in user of the workstation; receiving, from the radiology database, data related to the radiological imaging examination; identifying at least one patient episode context using both the received logged in user data and the received radiological imaging examination data; determining at least one suggested structured finding object (SFO) (60) for the identified at least one patient episode context; and displaying the at least one suggested SFO on the display during the entry of the medical report.

### FIELD

The following relates generally to the radiology arts, radiology reading arts, radiology workstation arts, radiology workstation user interfacing arts, and related arts.

### BACKGROUND

Radiology is a complex process involving several interacting medical professionals. In a typical sequence, a patient's physician orders a radiology examination. A radiology technician operates the imaging system, such as a magnetic resonance imaging (MRI) system, a computed tomography (CT) imaging system, a positron emission tomography (PET) imaging system, or so forth, or a combination of imaging system (e.g. PET/CT) to acquire images of an anatomical region of the patient in accordance with the physician's order. These images are stored in for example, a Picture Archiving and Communication System (PACS), Radiology Information System (RIS), or other form of healthcare database or computerized workstation and are later viewed, or “read”, by a radiologist, sometimes using a dedicated radiology workstation executing a radiology reading environment.

Radiology reading is a complex task, and is generally performed by highly skilled professionals, e.g. radiologists, with specialized training. The range of radiology examinations to be read may encompass numerous clinical tasks, e.g. health screenings, initial diagnoses, therapeutic assessments (e.g., whether oncology therapy is effectively controlling a malignancy), and so forth, and numerous medical conditions ranging from relatively simple bone fractures to complex oncology staging and tumor grading tasks. In many medical institutions, radiology is a high throughput department in which the radiologist is expected to perform many reading tasks per work shift. For example, a typical radiology department may expect the radiologist to perform an x-ray or ultrasound reading in a time frame of two minutes or less, while a more complex reading task such as a multi-slice CT or MRI may be expected to be performed in about five to seven minutes. In view of these considerations, it would be advantageous to provide tools for enhancing the efficiency and accuracy of the radiology examination reading process.

Improvements disclosed herein address the foregoing and other disadvantages of existing radiology reading systems, methods, and the like.

### BRIEF SUMMARY

In accordance with one illustrative example, a report-entry workstation includes a computer with a display, one or more user input devices, and at least one processor programmed to operate the computer to perform operations. The operations include: providing a user interface enabling a logged in user of the workstation to operate the one or more user input devices to retrieve and view, on the display, images of a radiological imaging examination stored in a radiology database and to operate the one or more user input devices to enter a medical report; receiving data related to the logged in user of the workstation; receiving, from the radiology database, data related to the radiological imaging examination; identifying at least one patient episode context using both the received logged in user data and the received radiological imaging examination data; determining at least one suggested structured finding object (SFO) for the identified at least one patient episode context; and displaying the at least one suggested SFO on the display during the entry of the medical report.

In accordance with another illustrative example, a non-transitory computer readable medium carrying software to control at least one processor to perform an image acquisition method is provided. The method includes: providing a user interface enabling a logged in user of the workstation to operate the one or more user input devices to retrieve and view, on at least one display, images of a radiological imaging examination stored in a radiology database and to operate the one or more user input devices to enter a medical report; receiving data related to the logged in user of the workstation; receiving, from the radiology database, data related to the radiological imaging examination; identifying at least one patient episode context using both the received logged in user data and the received radiological imaging examination data; determining at least one suggested structured finding object (SFO) for the identified at least one patient episode context; and displaying the at least one suggested SFO on the display during the entry of the medical report.

In accordance with another illustrative example, a report-entry workstation includes a computer including at least one display, one or more user input devices, and at least one processor programmed to operate the computer to perform operations including: providing a user interface enabling a logged in user of the workstation to operate the one or more user input devices to retrieve and view, on one of the first and second displays, images of a radiological imaging examination stored in a radiology database and to operate the one or more user input devices to enter a medical report; receiving data related to the logged in user of the workstation, the data including at least one of department information, role information, and title information; receiving, from the radiology database, data related to the radiological imaging examination, the data including at least one of modality, reason for exam, order information, and prior imaging sessions; identifying at least one patient episode context using both the received logged in user data and the received radiological imaging examination data; determining at least one suggested structured finding object (SFO) for the identified at least one patient episode context by generating natural language text by filling in one or more fields of a natural language template with values of dimensions of the SFO displaying the at least one suggested SFO on one of the first and second displays during the entry of the medical report; and enabling the logged in user to select a displayed suggested SFO using the one or more user input devices and adding one or more data entry fields defined by the selected suggested SFO to the medical report.

One advantage resides in expediting entry of image report findings.

Another advantage resides in organizing the radiology reading process around structured finding objects in order to facilitate collection and recordation of appropriate support for radiology findings, and to trigger secondary findings and appropriate use of third party tools.

Another advantage resides in providing a more effective and efficient radiology workstation user interface.

Further advantages of the present disclosure will be appreciated to those of ordinary skill in the art upon reading and understand the following detailed description. It will be appreciated that a given embodiment may provide none, one, two, or more of these advantages.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the invention.

FIG. 1 diagrammatically illustrates a report-entry system in accordance with one aspect.

FIG. 2 diagrammatically illustrates a radiology reading device supported by or incorporating a structured finding object (SFO)-based tool for facilitating collection and recordation of support for or characterization of radiological findings, triggering secondary findings, and interfacing with third party tools.

FIG. 3 diagrammatically illustrates a radiology reading task work flow suitably performed using the radiology reading device of FIG. 2.

### DETAILED DESCRIPTION

Structured reporting and structured findings provide a tool for expediting the radiology examination reporting process and improving its accuracy. It is recognized herein that a significant challenge to the adoption of structured reporting and the use of structured findings is how to properly model the current context to ensure the radiology workstation is suggesting the right structured finding descriptions (or “types”) at the right time. For example, one institution will likely have a different distribution in the types of findings that they image compared to another institution. Moreover, as recognized herein, the optimal finding suggestions are likely to be different depending upon the medical professional performing the reading, as well as depending upon the content of the radiology examination being read. For example, the reading of a radiology examination ordered by an oncologist may be more likely to include findings directed to tumor evolution; whereas, the reading of a radiology examination ordered by a general practice (GP) physician may be more likely to include diagnostic findings indicating the presence or absence of a particular type of tumor or malignancy.

The following is directed to providing contextual finding “type” recommendations for radiologists, oncologists, or others using a radiology workstation, oncology workstation, or the like which is connected to the Radiology Information System (RIS). Providing useful finding “type” recommendations depends strongly on the context of the workflow or patient episode, as for example different findings are likely to be made in an emergent care (emergency) situation as compared with a breast cancer screening session. These approaches may be implemented using structured finding objects (SFOs), which have fields for attributes such as the anatomy, the finding, and optional descriptors.

In the following, targeted SFO suggestions are made based on the combination of user context and examination context. A user context is drawn from information about the radiologist or other medical professional using the workstation. The user context may include various components. In the illustrative embodiments, the title and department of the medical professional are leveraged along with optionally other information to define a user context for the purpose of recommending finding types (e.g. SFOs).

Additionally, the examination context is drawn from DICOM metadata stored in the RIS, as well as patient information stored in the Electronic Health Record (ERR). Examination context may include information such as the reason for exam (if available), imaging modality, examination type, anatomical region, or so forth.

A workflow or patient episode context is drawn from the user context and the examination context. In one approach, this can be rule-based; alternatively the patient episode context can be output by a classifier learned from an annotated set of training cases. In some embodiments, two or more possible patient episode contexts may be output to accommodate uncertainty (for the purpose of generating finding type recommendations this is not overly problematic since it simply means generating a larger set of recommendations).

An SFO suggestion engine is used with the additional workflow or patient episode input. The output of the SFO suggestion engine is suitably a list of suggested SFOs which may be listed in a separate window on the workstation display or superimposed on a displayed image. If a listed SFO is selected (e.g. by a mouse click), the SFO form with user entry fields is inserted into the report being edited by the medical professional.

The disclosed SFO suggestion workstation component addresses a significant problem recognized herein, namely, that the majority of findings and descriptors are only reported in particular workflows; workflows like emergent care, interventional care, continuous monitoring, diagnostic imaging, screening programs, and oncology care cycle. Each of these workflows correlates with distinct sets of structured findings, some of which are most common in, or even unique to, that specific workflow. Similarly, structured findings that are relevant to two different workflows often have differing levels of relevance depending on the workflow. By accurately recognizing the current workflow of the imaging study, the findings and descriptors that are more relevant to the context of current workflow can be prioritized in suggesting SFOs.

Disclosed radiology reading devices and methods utilize the concept of a structured finding object (SFO), which is a digital entity, preferably codified in a radiological ontology such as RadLex (promulgated by the Radiological Society of North America, RSNA) or Systematized Nomenclature of Medicine—Clinical Terms (SnoMed CT, promulgated by the College of American Pathologists, CAP). The SFO at least partially characterizes an image finding in a structured format using syntax such as Annotation Imaging Mark-up (AIM), which is a radiology domain-specific XML syntax. In some suitable embodiments, an SFO is represented by dimensions each codified using a <key, value> pair where “key” identifies the dimension and “value” is the value of the dimension for the radiological finding in the current reading task. Dimensions are typically typed, and the value for a given dimension may itself be a complex (e.g. hierarchical) data structure.

By way of non-limiting illustration, some possible SFO dimensions are as follows. A “diagnosis” dimension may assume a string value from a set of possible diagnoses chosen from the radiology ontology such as “lesion”, “nodule”, “cyst”, “metastatic lesion”, “tumor” or so forth, and optionally having additional parameters such as a likelihood or probability value in the range [0,1] or having discrete string values such as “likely”, “unlikely”, or “presumably”. A “spatial” dimension may have a value comprising an ordered triplet of integers or real numbers representing the size in the X, Y and Z directions of a finding (e.g. tumor). The “spatial” dimension may optionally also assume a special value such as “absent” or the special triplet “0,0,0” for annotating a negative finding, e.g. not finding a tumor at all.

Additionally or alternatively, a qualitative “size” dimension may be provided, e.g. assuming qualitative values such as “large” or “small”. An “anatomy/body part” dimension specifies an anatomical region and may assume string values from the ontology such as “kidney”, “left lung”, “liver”, or so forth. A “sub-location” dimension may further localize the anatomy, e.g. assuming values such as “renal cortex”, “left lower lobe”, “liver segment 1”, or so forth. The set of allowable values for the “sub-location” dimension depends upon the “anatomy/body part” dimension. A “temporal” dimension may assume values such as “stable”, “interval increase in size”, or so forth. To assign a value for the “temporal” dimension the radiologist may need to refer back to a past radiology examination of the patient, e.g. to assess how the tumor size has changed since that last examination. A “plurality” dimension may assume values such as “one”, “multiple”, “various”, or so forth, and is for example used to specify the number of nodules observed in the image. Additional or other dimensions may be appropriate for a given type of finding, such as an “additional diagnostic” dimension which may assume a value such as “with calcifications” to further characterize a tumor. These are merely non-limiting illustrative dimensions for an SFO, and a SFO of a given type may have additional or other dimensions.

In general, a suggested SFO, when selected by the medical professional, initially provides an empty “template” which is the SFO data structure with dimensions appropriate for the particular finding being reported but with the values of those dimensions (at least mostly) blank. In some embodiments, some dimensions may be auto-populated based on information contained in the RIS, Electronic Medical Record (EMR), or other available database. Completion of SFO annotation results in a completed SFO in which many, and in some cases most or all, dimensions of the SFO are filled with values. The completed SFO may be converted to natural language text for inclusion in the radiology examination report, or may be included in another format, e.g. tabular or so forth.

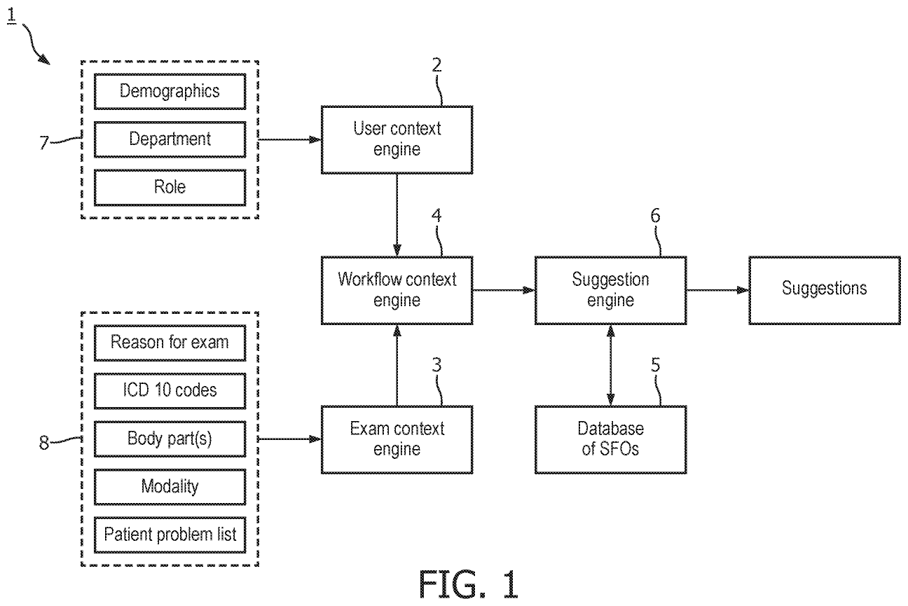

With reference now to FIG. 1, a schematic illustration of a contextual SFO suggestion sub-system 1 is shown, which may be a component of a radiology workstation or other reporting workstation. The contextual SFO suggestion sub-system 1 includes a user context engine 2, an exam context engine 3, a workflow context engine 4, a database of SFOs 5, and an SFO suggestion engine 6, each of these components being described in more detail below.

The user context engine 2, which can be implemented as a computer processor (e.g. the microprocessor and associated memory and/or other computational components of a radiology workstation), is programmed to collect data points about a user (i.e., a radiologist or a technician) relevant to a current patient episode context. For example, the user context engine 2 is programmed to collect the data points about the user from a first database 7. The data points can include information about the user, including demographic information (age), department information (abdominal, lung screening, mammography, etc.), role information (attending, fellow, etc.), title information, and so forth. Once retrieved, the user context engine 2 is programmed to aggregate these features into a vector, which is then transmitted to the workflow context engine 4.

The exam context engine 3, which can be implemented as a computer processor (e.g. the microprocessor and associated memory and/or other computational components of a radiology workstation), is programmed to collect data points about a current imaging exam relevant to the current patient care context. For example, the exam context engine 3 is programmed to collect data points about the current imaging exam from a second database 8 (or alternatively, from the first database 7). In some examples, the second database 8 can be a radiology information system (RIS) database or an electronic medical record (EMR) database. The exam context data points can include information about the current imaging exam, such as modality, reason for exam, order information, prior reports, prior imaging sessions, and so forth. Once retrieved, the exam context engine 3 is programmed to correlate and aggregate these features into a vector, which is then transmitted to the workflow context engine 4.

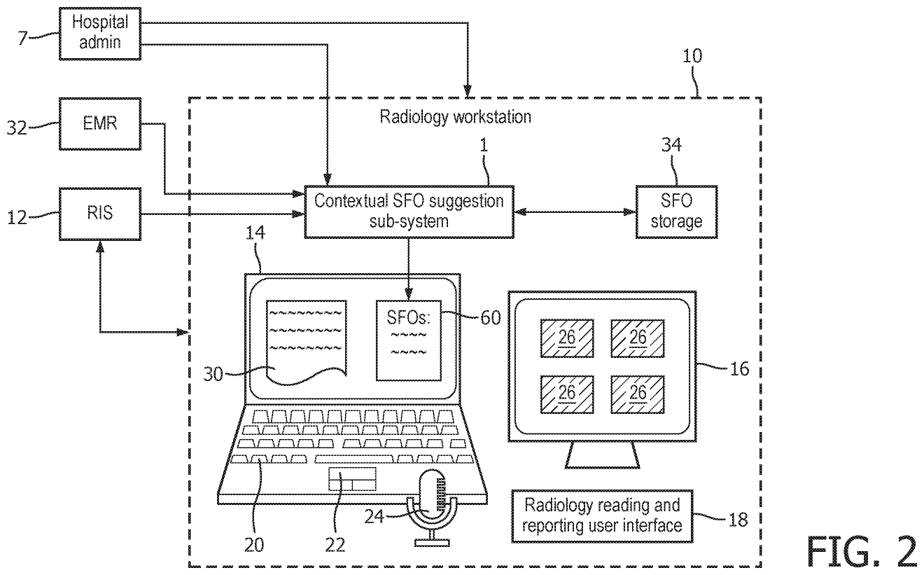

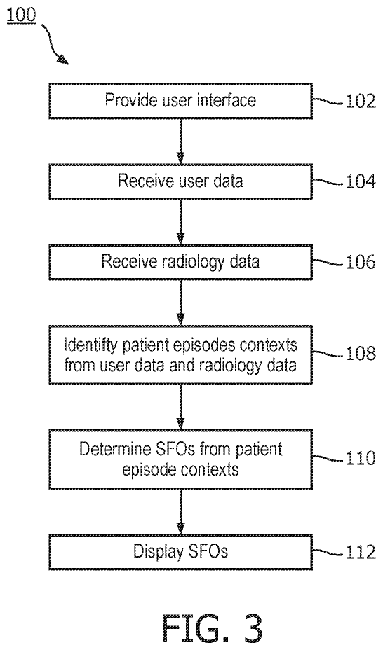

In one example, the workflow context engine 4 can be implemented as a computer processor (e.g. the microprocessor and associated memory and/or other computational components of a radiology workstation). The workflow context engine 4 is programmed to receive the output vectors from the user context engine 2 and the exam context engine 3. The workflow context engine 4 is then programmed to determine a possible workflow (also referred to herein as “patient episode context) of a predetermined set of workflow types of which the current imaging exam is a part. For example, the set of possible workflow types includes: tumor staging, lung cancer screening, breast cancer screening, emergent care, interventional cardiology, diagnostic imaging, and so forth. The workflow context engine 4 is programmed to use a set of manually curated rules to determine whether an input from the user context engine 2 and/or the exam context engine 3 (e.g., Reason for exam: “fever of unknown origin”) indicates a specific patient episode context (e.g., “diagnostic imaging”). The workflow context engine 4 is then programmed to generate a list of possible, indicated workflows, which are displayed to the user. In another embodiment, instead of a rule-based computer processor, the workflow context engine 4 is programmed with a machine-learning classifier to determine the set of possible workflows. The database of SFOs 5 and the SFO suggestion engine 6 are described in more detail with reference to FIGS. 2 and 3 below.

With reference now to FIG. 2, the contextual SFO suggestion sub-system 1 from FIG. 1 is shown as a component of an illustrative radiology workstation 10 which has access to a Picture Archiving and Communication System (PACS) or other Radiology Information System (RIS) 12 that stores radiology images acquired by imaging devices (not shown) such as ultrasound, magnetic resonance imaging (MRI), a computed tomography (CT), positron emission tomography (PET), and/or other imaging systems, and/or by hybrid or combined imaging systems such as PET/CT imaging systems. The workstation 10 includes one or more display components, e.g. first and second illustrative displays 14, 16, and implements a radiology reading and reporting environment or user interface 18, which may for example comprise the Philips Intellispace PACS Enterprise radiology reading environment (available from Koninklijke Philips N.V., Eindhoven, the Netherlands). The RIS 12 which may be implemented on a server computer accessed via a wide area network (WAN), local area network (LAN), wireless local area network (WLAN), the Internet, or so forth. The radiology reading environment 18 also provides the radiologist with reporting tools via which the radiologist records the radiology examination report, e.g. using a standardized reporting template such as an RSNA radiology reporting template.

The radiology workstation 10 further includes one or more user input devices 20, 22, 24 via which the radiologist may interact with the radiology reading and reporting environment 18 (for example, in order to pan or zoom images). The illustrative user input devices include a keyboard 20, a track pad 22 (which could be replaced by another pointing device such as a mouse, trackball, touch-sensitive screen, or the like), and a dictation microphone 24 which may be used in conjunction with speech recognition software to dictate a report. In the illustrative example, radiology images 26 are displayed on the display 16 while a radiology examination report 30 being drafted by the medical professional is displayed on the display 14, but other arrangements are contemplated. While a single radiology workstation 10 is illustrated by way of example, it is to be understood that the radiology department of a sizable medical institution may include two, three, or more, or many radiology workstations each accessing the server-based radiology reading and reporting environment and each either running its own instance of the reading/reporting environment or each accessing a common server-based reporting environment.

To start a radiology examination reading session, a user (such as a radiologist, oncologist, or the like) logs onto the workstation 10. This typically entails entering a username uniquely identifying the user, and providing a password via the keyboard 20 or other authentication (e.g. using a fingerprint reader, not shown). The user credentials (username and authentication information) are authenticated against information stored in a hospital administration database 7 (which may be the first database 7 of FIG. 1). After the authentication, user information may be retrieved by the contextual SFO suggestion sub-system 1 of the workstation 10, i.e. more specifically by the user context engine 2 (see FIG. 1). Typically, this includes identifying user's title and department information. After completing the logon/authentication process, the workstation 10 provides the user interface 18 enabling the logged-in user of the workstation 10 to operate the one or more user input devices 20, 22, 24 to retrieve and view, on the display 16, images of a radiological imaging examination stored in a radiology database (12) and to operate the one or more user input devices 20, 22, 24 to enter the medical report 30.

As the logged-in user performs the radiology examination reading, the contextual SFO suggestion sub-system 1 receives, from the RIS, PACS, or other radiology database 12 or from an Electronic Medical Record (EMR) or other patient database 32, data related to the radiological imaging examination being read, e.g. via the examination content engine 3 (see FIG. 1). The contextual SFO suggestion sub-system 1 determines at least one patient episode context using both the logged-in user data received from the hospital administration database 7 and the radiological imaging examination data received from the RIS 8 and optionally other databases such as the illustrative EMR 32. In the expanded view of FIG. 1, the workflow context engine 4 makes the determination of the patient episode context, as already described, and the suggestion engine 6 determines at least one suggested SFO 60 for the identified at least one patient episode context. The at least one suggested SFO 60 is suitably displayed on the display 14 during the entry of the medical report 30, e.g. in a separate window on the display 14 (or, alternatively, on the second display 16). Optionally, the SFOs associated with each patient episode context are sorted, e.g. the most likely findings being listed at top. If two or more patient episode contexts are identified, then the combined SFO list may similarly be sorted based on the most likely patient episode context.

If the user clicks on a suggested SFO of the list of suggested SFOs 60, then the template of the SFO is retrieved from an SFO database 34 and inserted into the radiology examination report 30. In a preferred embodiment, the template is rendered in the report 30 as natural language text (e.g. full sentences, bullet items, or so forth) with a data entry field for each <key, value> pair of the SFO (where again “key” identifies a dimension and “value” is the value of the dimension). For example, if the key is “tumor size” then the inserted text may comprise: “Tumor size is ______ mm” where the underscore indicates a user-entry field and “mm” denotes the unit of measurement (millimeters). It will be appreciated that the suggested SFOs 60 can also be stored in the SFO database 34 (or any other suitable database). Advantageously, the suggested SFOs 60 can be accessed by other users, such as a downstream user (i.e., a doctor or a nurse) or a third party user (i.e., a specialist at another health care facility).

In a typical radiology reading environment, a radiologist logs onto the workstation 10 and may then read a large number of radiology examinations over the course of a work shift. Advantageously, the user context remains the same for all such readings since the logged-in radiologist is the same for all these readings (until the radiologist logs out). Thus, the user context engine 2 (see FIG. 1) need execute only once, upon login of the radiologist (or other user). On the other hand, the examination context engine 3 runs each time a new radiology examination is retrieved for reading. In embodiments in which the workflow context engine 4 is rules-based, the rules for identifying a particular workflow (i.e. patient episode context) may be advantageously grouped by user context, so that as each new radiology examination is loaded for reading only the sub-set of rules associated with the user context of the currently logged-in user are searched to identify possible patient episode context(s).

Furthermore, it is contemplated for the suggested SFOs 60 to be filtered and/or sorted by other criteria beyond the patient episode context derived from the user and examination contexts. For example, the radiology examination report 30 being entered can be mined by keyword searching, natural language processing techniques, or the like to identify a finding that is already reported in the examination report 30 (for example, entered manually rather than using an SFO). If this finding corresponds to one of the suggested SFOs 60 then that SFO suggestion is suitably removed from the list. As another example of additional filtering, if the user selects one of the suggested SFOs and it is thereby imported into the radiology examination report 30, then that selected SFO is removed from the list of suggested SFOs 60 and, moreover, any remaining findings of the first of SFOs 60 that are inconsistent with the selected SFO are also removed. (For example, if the user selects a suggested “tumor size” SFO then another suggested finding “No tumor observed” is inconsistent with this selected finding and is removed).

With reference now to FIG. 3, an SFO selection method 100 is shown. At step 102, the user interface 18 is provided to enable a logged in user of the workstation to operate the one or more user input devices to retrieve and view, on the display 14, 16, images 26 of a radiological imaging examination stored in the radiology database 12 and to operate the one or more user input devices to enter the medical report 30. At step 104, data related to the logged in user of the workstation 10 (e.g., at least one of department information, role information, and title information) is received (e.g., from the first database 7). At step 106, data related to the radiological imaging examination (e.g., at least one of modality, reason for exam, order information, and prior imaging sessions) is received (e.g., from the PACS or RIS 12). At step 108, at least one patient episode context is identified using both the received logged in user data and the received radiological imaging examination data. At step 110, at least one suggested structured finding object (SFO) 60 is determined for the identified at least one patient episode context. At step 112, the at least one suggested SFO is displayed on the display 14, 16 during the entry of the medical report 30. It will be appreciated that these steps 102-112 may be performed in any suitable order. For example, the radiological imaging examination data (i.e, step 106) may be received before the user data (i.e. step 104) is received.

It will be appreciated that the illustrative computational, data processing or data interfacing components of the report-entry and SFO-based tools, e.g. components 1, 2, 3, 4, may be embodied as a non-transitory storage medium storing instructions executable by an electronic processor (e.g. the sub-system 1 or the computer of the workstation 10) to perform the disclosed operations. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A report-entry workstation comprising:
a computer including a display, one or more user input devices, and at least one processor programmed to operate the computer to perform operations including:
providing a user interface enabling a logged in user of the workstation to operate the one or more user input devices to retrieve and view, on the display, images of a radiological imaging examination stored in a radiology database and to operate the one or more user input devices to enter a medical report;
receiving data related to the logged in user of the workstation;
receiving, from the radiology database, data related to the radiological imaging examination;
identifying at least one patient episode context using both the received logged in user data and the received radiological imaging examination data;
determining at least one suggested structured finding object (SFO) for the identified at least one patient episode context;
displaying the at least one suggested SFO on the display during the entry of the medical reported; and
enabling the logged in user to select a displayed suggest SFO using the one or more user input devices and adding one or more data entry fields defined by the selected suggested SFO to the medical report.

2. (canceled)

3. The report-entry workstation according to claim 1, wherein:
the SFO is selected based on the identified patient episode context at least by defining <key, value> pairs in which the key denotes a dimension of the SFO representing information supporting or characterizing the identified patient episode context and the value denotes a value for the dimension; and
the selected SFO based on the patient episode context is built at least in part by receiving values for dimensions of the retrieved SFO using the at least one user input device.

4. The report-entry workstation according to claim 1, wherein the SFOs are structured Annotation Imaging Mark-up (AIM) objects.

5. The report-entry workstation according to claim 1 wherein each SFO defines <key, value> pairs in which the value is configured to assume values only from a set of possible values chosen from the current image acquisition session.

6. The report-entry workstation according to claim 1 wherein the at least one processor is further programmed to:
generate natural language text by filling in one or more fields of a natural language template with values of dimensions of the SFO.

7. The report-entry workstation according to claim 1, wherein the at least one processor is further programmed to:
complete annotation data entry fields of the medical report with values for dimensions of the retrieved SFO.

8. The report-entry workstation according to claim 1, wherein the images are displayed on a first display, the medical report is displayed on a second display, and the selected SFO is displayed on one of the first and second displays.

9. The report-entry workstation according to claim 1, wherein at least one of:
the data related to a user of the workstation includes at least one of department information, role information, and title information; and
the data related to a current image acquisition session includes at least one of modality, reason for exam, order information, and prior imaging sessions.

10. A non-transitory computer readable medium carrying software to control at least one processor to perform an image acquisition method, the method including:
providing a user interface enabling a logged in user of the workstation to operate the one or more user input devices to retrieve and view, on at least one display, images of a radiological imaging examination stored in a radiology database and to operate one or more user input devices to enter a medical report;
receiving data related to the logged in user of the workstation;
receiving, from the radiology database, data related to the radiological imaging examination;
identifying at least one patient episode context using both the received logged in user data and the received radiological imaging examination data;
determining at least one suggested structured finding object (SFO) for the identified at least one patient episode context;
displaying the at least one suggested SFO on the display during the entry of the medical report; and
enabling the logged in user to select a displayed suggested SFO using the one or more user input devices and adding one or more data entry fields defined by the selected suggested SFO to the medical report.

11. (canceled)

12. The non-transitory computer readable medium according to claim 1, wherein:
the SFO is selected based on the identified patient episode context at least by defining <key, value> pairs in which the key denotes a dimension of the SFO representing information supporting or characterizing the identified patient episode context and the value denotes a value for the dimension; and
the selected SFO based on the patient episode context is built at least in part by receiving values for dimensions of the retrieved SFO using the at least one user input device.

13. The non-transitory computer readable medium according to claim 1, wherein each SFO defines <key, value> pairs in which the value is configured to assume values only from a set of possible values chosen from the current image acquisition session.

14. The non-transitory computer readable medium according to claim 1, wherein the method further includes:
generating natural language text by filling in one or more fields of a natural language template with values of dimensions of the SFO.

15. The non-transitory computer readable medium according to claim 1, wherein the method further includes:
completing annotation data entry fields of the medical report with values for dimensions of the retrieved SFO.

16. The non-transitory computer readable medium according to claim 1, wherein the images are displayed on a first display, the medical report is displayed on a second display, and the selected SFO is displayed on one of the first and second displays.

17. The non-transitory computer readable medium according to claim 1, wherein at least one of:
the data related to a user of the workstation includes at least one of department information, role information, and title information; and
the data related to a current image acquisition session includes at least one of modality, reason for exam, order information, and prior imaging sessions.

18. (canceled)

19. (canceled)

20. (canceled)
